(19) United States
(12) Patent Application Publication
(10) Pub. No.: US 2016/0138071 A1
(43) Pub. Date: May 19, 2016

(54) METHODS FOR DETECTING THE PRESENCE OF MICROBES

(71) Applicant: PILOT POINT LLC, Sarasota, FL (US)

(72) Inventor: Stephen C. Edberg, Longboat Key, FL (US)

(73) Assignee: Pilots Point LLC, Sarasota, FL (US)

(21) Appl. No.: 14/898,374

(22) PCT Filed: Jun. 16, 2014
(86) PCT No.: PCT/US14/42539

§ 371 Date: Dec. 14, 2015

Related U.S. Application Data
(60) Provisional application No. 61/835,197, filed on Jun. 14, 2013.

(51) Int. Cl. C12Q 1/06; G01N 35/00; G01N 21/49; G01N 35/04; G01N 21/25; C12Q 1/04; G01N 35/02

(52) U.S. Cl. CPC C12Q 1/06; C12Q 1/04; G01N 35/00871; G01N 35/025; G01N 35/04; G01N 21/251; G01N 21/49; G01N 2035/00891; G01N 2035/0401; G01N 2201/061

(57) ABSTRACT

A method and instrument for detecting the presence or absence of a target organism in a test sample is provided. The instrument includes a carousel, a light source, alight detector, and a control system. The carousel is configured to hold a plurality of test sample containers, with each container held by the carousel at a container position. Each of the containers holds an admixture of a test mixture inoculated with the test sample. The light detector is operable to detect light emitted by the light source that has passed through the test sample disposed within the test container. The control system includes a processor. The control system is adapted to produce information indicative of the presence or absence of the target organism in the test sample using signals produced by the light detector.

This application claims the benefit of U.S. Provisional Patent Application Ser. No. 61/835,197 filed Jun. 14, 2013.

### BACKGROUND OF THE INVENTION

1. Technical Field

The present invention generally relates to methods and apparatus for detecting microbes, and more particularly relates to methods and apparatus for detecting the absence or presence of a target microbe in a biological, environmental, or food sample.

2. Background Information

The bacterium Staphylococcus aureus (S. aureus) may be transmitted between healthy individuals by skin to skin contact, or from a commonly shared item or a surface (e.g., tanning beds, gym equipment, food handling equipment, etc.) where the transfer may be made to a subsequent person who uses the shared item or touches the surface. Of great medical concern is the recognition that healthy people entering hospitals may “carry” S. aureus (e.g., on their skin, or in their noses, etc.) without any signs or symptoms that they do so. In the presence of favorable conditions (often found in but not limited to hospitals), the S. aureus can activate and cause serious infection. In addition, S. aureus can also be a source of food poisoning, often caused by a food handler contaminating the food product (e.g., meat, poultry, eggs, salads containing mayonnaise, bakery products, dairy products, etc.)

There are two categories of S. aureus based on an individual clone's susceptibility to the class of antibiotics that began with methicillin. These are methicillin susceptible S. aureus (MSSA), and methicillin resistant S. aureus (MRSA). Until only a few years ago, MRSA was most commonly found in hospitals. Now, many are also present in the noses, skin, etc. of people in the non-hospital community. Moreover, these MRSA are increasingly causing serious infections in the community. MRSA is particularly serious because very few antibiotics (e.g., vancomycin) have been shown to be uniformly effective against MRSA.

The Center for Disease Control and Prevention actively surveys for the development of methicillin resistant S. aureus. In 2000, the Society for Healthcare of America guidelines recommended contact isolation for patients with MRSA. In addition to the morbidity and mortality caused by MRSA, it has been estimated that each case of infection costs at least $23,000. Accordingly, many hospitals and nursing homes proactively sample patients for MRSA [Clany, M., Active Screening in High-Risk units is an effective and cost-avoidant method to reduce the rate of methicillin-resistant Staphylococcus aureus infection in the hospital, Infection Control and Hospital| 27:1009-1017, 2006].

A large number of classical culturing procedures are utilized to detect MSSA and MRSA from human, animal, food, etc. samples. These tests typically require the sample to be cultured, isolates removed, and then tested to verify the presence or absence of S. aureus. These procedures can be time consuming and must be performed by a skilled technician. Likewise, there are diagnostic and epidemiological screening tests for a wide variety of bacteria. These include vancomycin resistant enterococcus (VRE), vancomycin resistant Staphylococcus aureus (VRSA), vancomycin intermediate Staphylococcus aureus (VISA), carbapenamase resistant enterobacteriacae (CRE, and known as a subset KPC), ciprofloxacin resistant enterobacteriacae, and others.

It would, therefore, be desirable to provide a test instrument and method that can more rapidly detect a target bacteria such as S. aureus directly from a sample, one that does not require a skilled technician to perform the method, one that can be performed without the need to develop isolates from the specimen (i.e., one that can be performed on a “first generational” sample), and one that does not require a large concentration of target organisms to be accurate.

### SUMMARY OF THE INVENTION

According to an aspect of the present invention, an instrument for detecting the presence or absence of a target organism in a test sample is provided. The instrument includes a carousel, a light source, a light detector, and a control system. The carousel is configured to hold a plurality of test sample containers, with each container held by the carousel at a container position. Each of the containers is operable to hold an admixture of a test mixture inoculated with the test sample. The light source is operable to emit light along a plurality of different wavelengths. The light detector is operable to detect the light emitted by the light source. The light source and light detector are relatively positioned so that light emitted by the light source travels along a light axis extending through the test sample disposed within the test container and is detected by the light detector. The light detector is operable to produce signals representative of the light detected by the light detector. The control system includes a processor. The control system is adapted to produce information indicative of the presence or absence of the target organism in the test sample using signals produced by the light detector.

In an embodiment of the foregoing aspect the light source emits ultraviolet light.

In a further embodiment of any embodiment or aspect above, the light source emits infrared light.

In a further embodiment of any embodiment or aspect above, the light source emits white light.

In a further embodiment of any embodiment or aspect above, the control system is adapted to determine a change of color in the admixture from the signals produced by the light detector.

In a further embodiment of any embodiment or aspect above, the control system is adapted to determine a change in a level of turbidity within the admixture from the signals produced by the light detector.

In a further embodiment of any embodiment or aspect above, the light source is disposed on one side of the carousel positions and the light detector is positioned on the opposite side of the carousel positions.

In a further embodiment of any embodiment or aspect above, the light source and light detector are stationary, and the carousel is movable relative to the light source and light detector so that each container position is alignable with the light source and light detector.

In a further embodiment of any embodiment or aspect above, the carousel is adapted for rotational movement.

In a further embodiment of any embodiment or aspect above, the carousel is adapted for linear movement.

In a further embodiment of any embodiment or aspect above, the carousel is stationary and the light source and light detector are movable relative to the carousel so that each container position is alignable with the light source and light detector.

In a further embodiment of any embodiment or aspect above, the carousel includes “n” number of container positions, where “n” is an integer, and the instrument includes “n” light source/light detector pairs, each light source/light detector pair positioned so that light emitted by the light source of the pair travels along a light axis extending through the test sample disposed within the test container and is detected by the light detector of the pair, and the carousel, the light source, and the light detector are stationary relative to one another.

In a further embodiment of any embodiment or aspect above, the control system is adapted to control the light source to emit light along the test axis for each container holding test sample and to receive the signals representative of the emitted light passing through the container and sample from the light detector at periodic intervals of time over a test period.

In a further embodiment of any embodiment or aspect above, the control system is adapted to produce the information indicative of the presence or absence of the target organism for a given one of the test samples for each periodic interval of time.

In a further embodiment of any embodiment or aspect above, the information indicative of the presence or absence of the target organism for a given one of the test samples for each periodic interval of time includes a quantifiable value representative of the per volume number of CFUs of the target organism within the test sample.

In a further embodiment of any embodiment or aspect above, the control system is adapted to create a growth curve for the test period using the information indicative of the presence or absence of the target organism for the given one of the test samples at each periodic interval of time.

In a further embodiment of any embodiment or aspect above, the control system is adapted to display the growth curve on a monitor.

In a further embodiment of any embodiment or aspect above, the information indicative of the presence or absence of the target organism in the test sample includes a quantifiable value representative of the per volume number of CFUs of the target organism within the test sample.

According to another aspect of the present invention, a method for detecting the presence or absence of a target organism in a test sample is provided. The method includes the steps of: a) providing an instrument having a carousel configured to hold a plurality of test sample containers, with each container held by the carousel at a container position, and each of which containers is operable to hold an admixture of a test mixture inoculated with the test sample, a light source and a light detector paired together, and a control system; b) emitting white light using the light source, and detecting light emitted by the light source using the light detector, which light has traveled along a light axis extending through the test sample disposed within the test container, which light detector is operable to produce signals representative of the light detected by the light detector; and c) producing information indicative of the presence or absence of the target organism in the test sample using a control system to process the signals produced by the light detector.

In a further embodiment of any embodiment or aspect above, the step of producing information includes at least one of determining a change of color in the admixture or determining a change in a level of turbidity within the admixture.

In a further embodiment of any embodiment or aspect above, the method further includes the step of moving one of the carousel and the light source and light detector pair, relative to the other, so that each container position is alignable with the light source and light detector.

In a further embodiment of any embodiment or aspect above, the step of emitting white light using the light source, and detecting light emitted by the light source is performed at periodic intervals of time over a test period.

In a further embodiment of any embodiment or aspect above, the step of producing information is performed for each periodic interval of time.

In a further embodiment of any embodiment or aspect above, the step of producing information includes producing a quantifiable value representative of the per volume number of CFUs of the target organism within the test sample.

In a further embodiment of any embodiment or aspect above, the step of producing a growth curve for the test period using the information produced at each periodic interval of time.

These and other objects, features and advantages of the present invention will become more apparent in light of the following detailed description of preferred embodiments thereof, as illustrated in the accompanying drawings.

### BRIEF DESCRIPTION OF THE DRAWINGS

FIG. 1 is a diagrammatic view of an embodiment of the present instrument.

FIG. 2 is a diagrammatic view of a test container containing a hydrated test sample.

FIG. 3 is a diagrammatic view of the test container, illustrating a swab containing a test sample being inserted into the test container.

FIG. 4 is a diagrammatic view of the test container, illustrating a swab containing a test sample inoculating the test sample.

FIG. 5 is a diagrammatic view of an embodiment of the present instrument.

FIG. 6 is a diagrammatic top view of an embodiment of the present instrument.

FIG. 7 is a diagrammatic view of an embodiment of the present instrument.

FIG. 8 is a graph illustrating light absorption as a function of wavelength.

FIG. 9 is a graph showing signal magnitude per volume as a function of incubation time, with growth curves depicted in the chart, for a given type of test mixture.

FIG. 10 is a graph showing signal magnitude per volume as a function of incubation time, with growth curves depicted in the chart, for a given type of test mixture and a plurality of different MRSA isolates.

FIG. 11 is a graph showing signal magnitude per volume as a function of incubation time, with growth curves depicted in the chart, for a given type of test mixture.

FIG. 12 is a graph showing signal magnitude per volume as a function of incubation time, with growth curves depicted in the chart, for a given type of test mixture.

### DETAILED DESCRIPTION OF THE INVENTION

The present invention is directed to an instrument and method operable to be used with a plurality of microbiology detection tests. These tests include, but are not limited to, the AureusAlert® test (Pilots Point Holdings, LLC of Sarasota, Fla., USA), for the determination of all Staphylococcus aureus (e.g., Methicillin sensitive S. aureus (“MSSA”), Methicillin resistant S. aureus (“MRSA”), etc.) from a sample collected from a human (e.g., a nose or skin swab), or a sample collected from an environmental surface (e.g., surfaces located in hotels, spas, health clubs, beauty parlors, etc.), and the EPI-V™ test (Pilots Point Holdings, LLC of Sarasota, Fla., USA) which determines Vancomycin-resistant enterococcus from, for example, a stool sample. Other non-limiting examples of microbiology detection tests that can be used with the present invention include tests for carbapenamase resistant enterobacteriaceae, vancomycin intermediate Staphylococcus aureus, and ciprofloxacin resistant enterobacteriaceae. The present instrument is operable to perform all these tests. Embodiments of the AureusAlert® test are described in U.S. Pat. No. 8,268,579, which patent is hereby incorporated by reference in its entirety. Embodiments of the EPI-V test are described in U.S. Pat. No. 6,355,449, which patent is hereby incorporated by reference in its entirety. The present instrument is not limited to use with these types of tests, however.

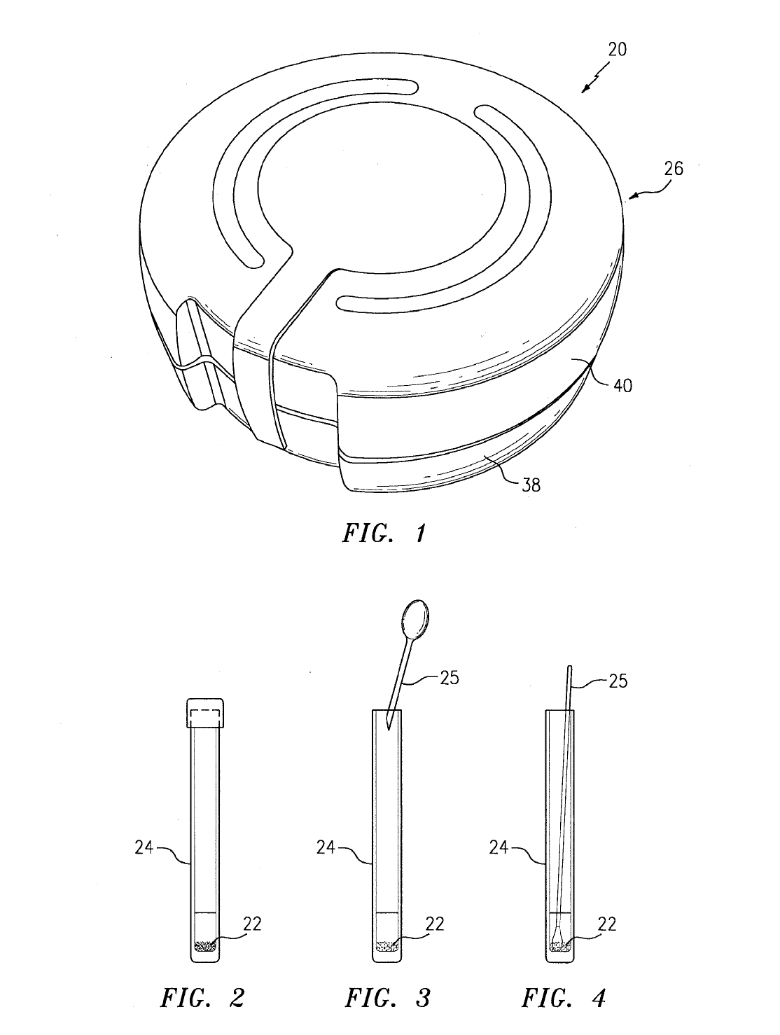

Now referring to FIGS. 1-6, the microbiology tests that can be used with the present instrument 20 utilize a stable test mixture 22 that can be disposed in a container 24 such as that shown in FIG. 2. A non-limiting example of a test mixture that can be used with the present instrument is a powdered test mixture that can be deposited in a test tube type container and subsequently hydrated. The present invention is not limited for use with any particular test mixture or with any particular type of sample container, other than the test container must be transparent. In fact, the present invention can be adapted for use with a variety of different test containers, types of tests, and test volumes.

The instrument 20 includes a housing 26, a carousel 28, a carousel drive mechanism 30, a light source 32, a light detector 34, and a control system 36.

The housing 26 includes a base 38 and a cover 40 (see FIG. 1). The cover 40 may be hingedly or removably attached to the base 38. The housing 26 includes structure 42 (e.g., bearings for pivotal or linear carousel movement, guides, etc.) for mounting the carousel 28, the light source 32, and the light detector 34. As will be explained below, the structure 42 for mounting the carousel 28, the light source 32, and the light detector 34 may be configured so that the carousel 28 is movable relative to the light source 32 and light detector 34, or the light source 32 and light detector 34 may be movable relative to the carousel 28, or both the light source 32/light detector 34 pair and the carousel 28 may be positionally fixed within the housing.

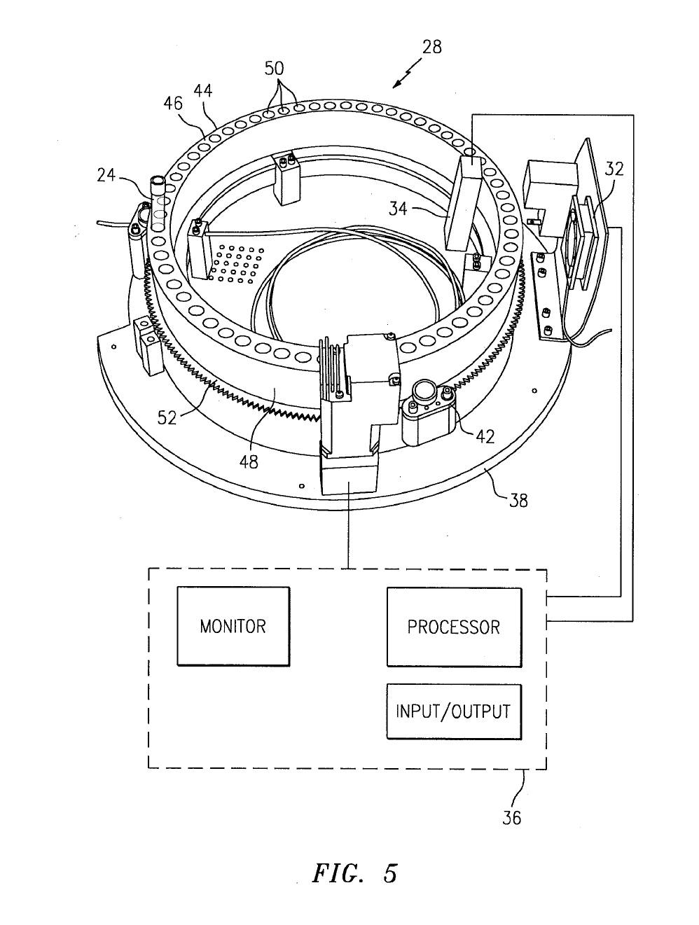

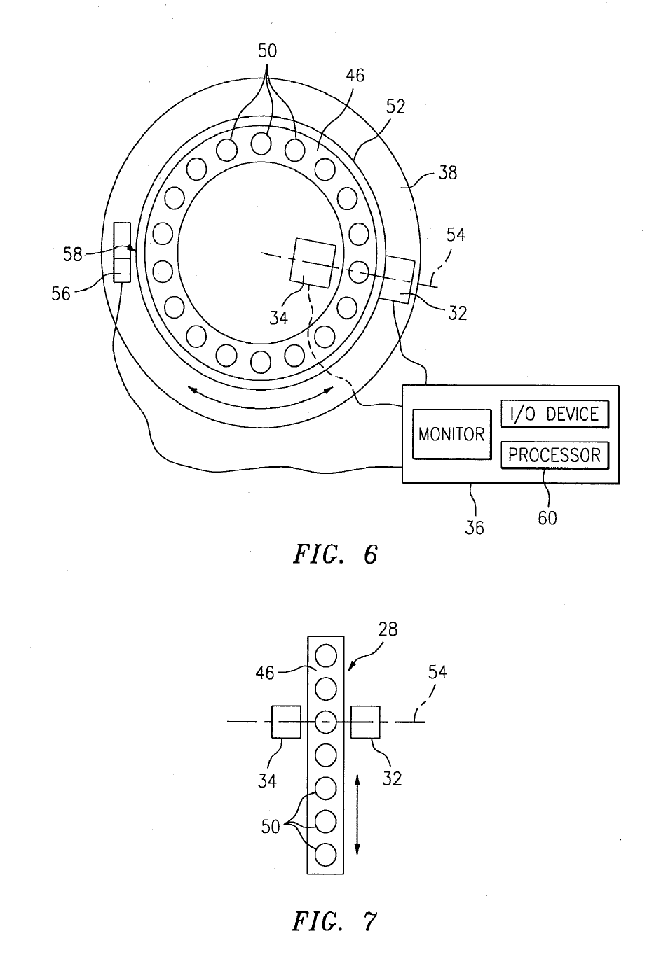

In a first embodiment, the carousel 28 includes a ring-shaped body 44 with an outside wall extending between a top surface 46 and a base flange 48. The carousel 28 is configured to hold a plurality of sample containers 24 at positions around the circumference of the ring-shaped body 44. For example in the carousel 28 embodiment shown in FIGS. 5 and 6, the ring-shaped body 44 is a solid ring with a plurality of cavities 50 disposed in the top surface 46, spaced around the circumference of the ring-shaped body 44. Each cavity 50 is configured to receive and hold a sample container 24 (e.g., a tube) in a vertical orientation. Each cavity 50 is configured to hold a container 24 securely within the carousel 28, but in a manner such that an adequate amount of typical sample volume within each container 24 is visible above the top surface 46 of the body 44. In the embodiment shown in FIGS. 5 and 6, the carousel 28 includes a drive flange 52 extending radially outwardly from the side wall of the body 44. As will be explained below, the drive flange 52 cooperates with the carousel drive mechanism 30 to enable rotation of the carousel 28. As indicated above, the housing 26 includes structure 42 for mounting the carousel 28 for rotational movement of the carousel 28 relative to the housing 26. The carousel 28 may include structure that cooperates with the housing structure 42 to facilitate the rotational movement; e.g., the housing 26 may include a male component received within a mating female component attached to, or formed in, the carousel 28 (or vice versa). The present instrument is not limited to the above described embodiment of the carousel 28.

The carousel drive mechanism 30 is operable to selectively rotate the carousel 28. As will be explained below, in this embodiment the light source 32 and light detector 34 are positioned at a fixed position relative to the carousel 28. The light source 32 produces light centrally aligned along a line referred to hereinafter as a “light axis” 54. The light detector 34 is positioned to receive light traveling along the light axis 54. The light axis 54 is typically oriented perpendicular to the portion of the carousel body 44 disposed between the light source 32 and the light detector 34 so that the light axis 54 will extend through substantially all of the depth of the sample admixture within the container 24 as will be explained below. The carousel drive mechanism 30 is operable to rotate the carousel 28 in a manner such that each cavity 50 disposed in the carousel 28 (and the container 24 residing in the cavity 50) may be selectively rotated into alignment with the light axis 54. The carousel drive mechanism 30 may be controlled to stepwise move the carousel 28 so that each cavity 50 is aligned with the light axis 54. Alternatively, the carousel drive mechanism 30 may be controlled to rotationally move the carousel 28 so that each cavity 50 rotates through the light axis 54; e.g., the carousel 28 moves continuously. In another embodiment, the carousel drive mechanism 30 may be controlled to selectively move the carousel 28 to positions where selected cavities 50 may be aligned with the light axis 54. For example, if the carousel 28 includes cavities one through sixty (1-60), the carousel drive mechanism 30 may be controlled to selectively move the carousel 28 such that cavity numbers ten (10), twenty (20), thirty (30), forty (40), fifty (50), and sixty (60), are consecutively brought into alignment with the light axis 54. This example represents only an example of how the carousel drive mechanism 30 may be controlled, and the carousel drive mechanism 30 is not limited thereto. The carousel drive mechanism 30 includes an electric motor 56 adequately sized to cause the carousel 28 to rotate, and a drive component 58. The drive component 58 operates as an interface between the motor 56 and the carousel 28. Examples of the drive component 58 include a gear or a pulley attached to a shaft of the motor 56, but the drive component 58 is not limited thereto. The carousel 28 example shown in FIG. 5 includes teeth disposed along the edge of the drive flange 52. The drive flange teeth may be engaged directly or indirectly with a mating drive component gear.

In alternative embodiments, the light source 32/light detector 34 pair may be moved relative to a stationary carousel 28, in which case a drive mechanism similar to that described above is used to move the light source 32/light detector 34 pair relative to the carousel 28. In other alternative embodiments, there may be a light source 32/light detector 34 pair for each cavity position along the carousel body 44, thereby obviating the need to move one of the carousel 28 and light source 32/light detector 34 pair relative to the other.

The light source 32 is operable to emit light along the light axis 54 at an intensity adequate for light not absorbed within the sample admixture to travel through sample admixture disposed in a container 24 seated in a cavity 50 of the carousel 28. In addition, or alternatively, the light source 32 is operable to emit light along the light axis 54 at a wavelength and intensity that permits the emitted light to act as an excitation light operable to cause fluorescent emission from materials present within an admixture of test sample and test mixture. In some embodiments, the light source 32 may emit white light (i.e., light containing substantially all the wavelengths of the visible spectrum at equal intensity) In some embodiments, the light source 32 may be configured to produce less than all the wavelengths of visible light. In some embodiments, the light source 32 may be configured to produce light at wavelengths in the ultraviolet range. In some embodiments, the light source 32 may be configured to produce light at wavelengths in the infrared range. The light source 32 can be controlled to selectively produce the light on demand. The light source 32 is not limited to any particular type, and examples include one or more light emitting diodes (LEDs), a fiber optic source, bulb light, etc.

An example of an acceptable light detector 34 is a charge couple device (CCD) type image sensor that converts light passing through the sample into an electronic data format. Complementary metal oxide semiconductors (“CMOS”) type image sensors are another example of a light detector 34 that can be used. The light detector 34 is operable to produce signals representative of the light received by the light detector 34.

In the embodiment shown in FIGS. 5-7, the light source 32 and the light detector 34 are aligned with one another along the light axis 54; i.e., the light detector 34 is positioned to receive light emitted by the light source 32 (or fluorescently emitted from the sample admixture) traveling along the light axis 54. The light detector 34 is disposed radially inside of the body 44 of the carousel 28 and the light source 32 is disposed radially outside of the body 44 of the carousel 28. The present apparatus is not limited to the above described light source 32/light detector 34 configuration; e.g., the light source 32 and light detector 34 may be positioned vice versa, or mirrors may be used to direct the light along the light axis 54.

The control system 36 can be used to control the operations described in association with any of the computer-implemented methods described herein. The control system 36 includes a processor 60 and a memory. In some embodiments, the control system 36 may further include one or both of a storage device and an input/output device. Each of the components may be interconnected using a system bus. The processor 60 is capable of processing instructions for execution within the system. The processor 60 is capable of processing instructions stored in the memory or on the storage device to display information (e.g., graphical information) for a user interface on the input/output device. The memory stores information within the system 36. In some embodiments, the memory is a computer readable medium. The memory can include volatile memory and/or non-volatile memory. In general, the storage device can include any non-transitory tangible media configured to store computer readable instructions. In one embodiment, the storage device is a computer-readable medium. The input/output device provides input/output operations for the system 36. In some embodiments, the input/output device may include a keyboard, a pointing device, a touch screen, or the like. In some embodiments, the input/output device includes a display unit for displaying graphical user interfaces. The features described can be implemented in digital electronic circuitry, or in computer hardware, firmware, or in combinations of them. The features can be implemented in a computer program product tangibly embodied in an information carrier, e.g., in a machine-readable storage device, for execution by a programmable processor; and features can be performed by a programmable processor executing a program of instructions to perform functions of the described embodiments by operating on input data and generating output.

In some embodiments, the control system 36 is an integral component within the instrument 20. In alternative embodiments, a portion or all of the control system 36 may be separable from the instrument 20. For example, the instrument 20 may be configured to communicate (e.g., via a USB port, wireless, or the like) to a computer (e.g., a laptop computer) having the described attributes of the control system 36. In this example, the instrument 20 may act as a peripheral device that can be connected to the computer. As another example, aspects of the control system 36 may be included directly within the instrument 20, and a computer can be placed in communication with the instrument and the computer used as an input/output device and as a monitor. The control system 36 is not limited to any of the described embodiments.

The computer system is in communication with the light source 32, light detector 34, and the carousel drive mechanism 30. The control system 36 is adapted (e.g., programmed) to control the operation of the light source 32, light detector 34, and the carousel drive mechanism 30. For example, the control system 36 is adapted to: 1) send and receive signals from the carousel drive mechanism 30 to position the carousel 28 (e.g., relative to the light axis 54); 2) send signals to the light source 32 to produce light; and 3) receive signals from the light detector 34.

Referring to FIG. 7, in another embodiment of the present invention, the instrument 20 includes a linearly-oriented carousel 28 (referred to hereinafter as a “linear carousel”) for holding the sample containers 24. For example, the linear carousel 28 in this embodiment has a linear-shaped body 44 (e.g., straight line) with a width, a length, and a height, first and second side walls extending heightwise between a top surface 46 and a base flange. The side walls also extend lengthwise between a first lengthwise end 62 and a second lengthwise end 64. The linear carousel 28 is configured to hold a plurality of sample containers 24 at positions along the length of the linear carousel 28. For example in the carousel 28 embodiment shown in FIG. 7, the body 44 is solid extending lengthwise along a straight line with a plurality of cavities 50 disposed in the top surface 46. Each cavity 50 is configured to receive and hold a sample container 24 (e.g., a tube) in a vertical orientation. Each cavity 50 is configured such that the container 24 is securely held within the body 44, but shallow enough such that an adequate amount of typical sample volume within each container 24 is visible above the top surface 46 of the body 44.

The linear carousel 28 may be moved relative to the light source 32 and light detector 34, or the light source 32 and detector may be moved along the length of the linear carousel 28. In some embodiments, there may be a light source 32/light detector 34 pair for each cavity position along the carousel body, thereby obviating the need to move the linear carousel 28 relative to a light source 32/light detector 34 pair, or vice versa. In those instances where one of the carousel 28 or a light source 32/light detector 34 pair is moved relative to the other, the housing 24 includes structure for mounting the moving part relative to the housing.

In embodiments that include a linear carousel 28 and one of the linear carousel 28 or a light source 32/light detector 34 pair is moved relative to the other, the carousel drive mechanism 30 is operable to selectively move the moving component relative to the stationary component. In a manner similar to that described above, in the linear carousel 28 embodiment the carousel drive mechanism 30 is operable to move the linear carousel 28 in a manner such that each cavity 50 disposed in the carousel 28 (and the container 24 residing in the cavity 50) may be selectively moved into alignment with the light axis 54; e.g., stepwise movement, continuous movement, or selective positioning. In those embodiments that include linear movement of the linear carousel 28 or the light source 32/light detector 34 pair, the carousel drive mechanism 30 includes an electric motor adequately sized to cause the moving component to linearly travel, and a drive component that interfaces with the moving component to accomplish the linear travel.

The present invention instrument and method are operable to interrogate test sample admixtures in an automated manner and produce results of the interrogations as a function of time.

In the operation of the instrument, the user prepares hydrated admixtures of test sample and text mixture disposed within a container 24. For example, for those test mixtures that require hydration, a predefined amount of water, or other acceptable medium, is added to the test mixture disposed within a tube. Once the test mixture and water are mixed within the tube, the admixture is inoculated with a test sample specimen. For example, a specimen collected via a nose swab 25 can be used to inoculate the test mixture by placing the swab 25 within the mixture as is shown in FIG. 4.

After the inoculation, the container 24 holding the inoculated admixture is placed in the carousel 28. For purposes of this explanation, it will be assumed that a container 24 is placed in every cavity 50 within the carousel 28, although that is not required. Once the carousel 28 is loaded, the housing cover 40 is placed over the carousel 28 and the testing is initiated.

The control system 36 may be programmed to search and identify the carousel position (and therefore the carousel cavities) so that the position/cavity number of any particular cavity 50 being interrogated is known.

The instrument 20 preferably includes a temperature control device that maintains the sample admixtures at a predetermined temperature for the duration of the test period. Some tests performed by the present instrument 20 may be performed at room temperature. In this case, the temperature control device may not operate to increase the temperature of the sample admixtures (e.g., the temperature of the environment within the instrument, which in turn dictates the temperature of the sample admixtures) but will sense and monitor the temperature to ensure the predetermined temperature is maintained constant during the test period. For those tests that require a temperature other than room temperature, the temperature control device may include a heating unit, a cooling unit, or both, that can establish and maintain the predetermined sample admixture temperature within the instrument during the incubation process.

During the incubation process, which may take place over an extended period of time (e.g., up to 24 hours), each container 24 is periodically interrogated with light from the light source 32. The light emitted from the light source 32 is directed through the container 24 and the sample admixture disposed therein, and light passing through (or emitted from) the same is collected by the light detector 34. The light detector 34, in turn, produces signals representative of the light collected and passes the signals onto the control system processor which subsequently processes the signals. The periodic rate at which each container 24 is interrogated by light may be selected to accommodate the testing at hand; e.g., light interrogation of each container 24 every fifteen (15) minutes provides useful information when testing most types of samples. The above described interrogation process includes aligning each container 24 along the light axis 54 at the periodic time interval; e.g., container 1 is aligned and interrogated, then the carousel 28 is moved (e.g., rotated or linearly moved) further until container 2 is aligned and interrogated, etc. As indicated above, in some embodiments, the carousel 28 may be stationary and the light source 32/light detector 34 pair is movable. In those instances, a similar process is followed except that it is the light source 32/light detector 34 pair that is periodically moved for interrogation. In those embodiments wherein the light source 32/light detector 34 pair(s) and the carousel 28 are stationary, a light source 32/light detector 34 pair is aligned with each carousel 28 container position; i.e., the light axis 54 passes through the container 24. In these stationary embodiments, the control system 36 may be adapted to operable to activate the appropriate light source 32/light detector 34 pairs in a manner that prevents cross talk of the interrogating light; e.g., the light source 32/light detector 34 pairs may be operated sequentially. As indicated above, in some embodiments, the carousel 28 (or the light source 32/light detector 34 pair) may be moved continuously. In these embodiments, the light source 32/light detector 34 pair interrogates each sample container 24 an amount of time adequate to produce the desired data.

The detected light signal data for each container 24 is stored/recorded for each container 24 as a function of time by the control system 36. For example, each sample container 24 is interrogated at the fifteen (15) minute interval and the light signal data is stored, and then the interrogation process is repeated at the thirty (30) minute interval, at the forty-five (45) minute interval, etc. By the end of the test, a data point associated with each periodic interrogation of a particular container 24 is available so that changes in the light properties (e.g., color and/or turbidity) of the sample can be evaluated over the incubation period.

The admixture of the test sample and test mixture will have an initial color and/or degree of transparency. Depending upon the presence or absence of a target organism within the test sample, the color and/or degree of transparency/opacity of the sample may change during the incubation period. For example, if the test sample contains the target organism, the test mixture will selectively promote the growth of the target organism within the admixture. In some instances, a test mixture may include a metabolizable substrate that causes a change in color within the admixture as the substrate is metabolized by the target organism. If the target organism is present within the sample, the change in color becomes increasing more apparent as a function of time within the incubation period; e.g., the admixture may initially have a first color, and over the incubation period, the admixture will change to a second color, the intensity of which is a function of the amount of target organism present within the test sample. In similar fashion, some test mixtures may indicate the presence of a target organism via the presence or absence of turbidity within the admixture. In these instances, if the target organism is present within the sample, the change in turbidity within the admixture becomes increasing more apparent as a function of time within the incubation period; e.g., the admixture may initially be substantially transparent, and over the incubation period, the admixture will change to an opaque/turbid mixture. The degree of turbidity/opacity is a function of the amount of target organism present within the test sample. Conversely, an admixture that does not change in color or opacity/turbidity would indicate a negative result; i.e., an indicator of the absence of the target organism within the test sample.

The degree to which an admixture of the test sample and test mixture may have a color and/or degree of transparency change can be evaluated relative to a negative control. For example, the control system 36 may be programmed to include color and/or transparency values representative of a negative control values (i.e., no target organism present) for each test contemplated. The negative control values can be stored within the control system 36 in a variety of different ways (e.g., look up tables, algorithmic solutions, etc.), and the control system is not limited to any particular type of access to such data. As part of the sample analysis, the color and/or transparency of the sample admixture can be comparatively analyzed relative to the negative control values to facilitate a determination of the presence or absence of a target organism within a sample.

The present instrument is operable to produce signal data results for a variety of test mixtures, which test mixtures may be specific to a variety of different organisms. The signals produced by the light source 32/light detector 34 pairs may be representative of light absorbed within the sample admixture. The difference in light intensity (i.e., the absorbance) between the incident light (Iₒ) and the sensed light (I) can be evaluated as a function of the Beer-Lambert law

$$A = - ln ( \frac{I}{{I}_{o}} )$$

which defines absorbance (“A”) as a function of incident light (Iₒ) and the sensed light (I), along a linear path through a medium (e.g., test sample admixture). Using the present invention, the absorbance is determined for light traveling along the light axis 54 through the sample admixture. Because the test containers 24 hold a significant volume of sample admixture and the depth of the sample along the light axis 54 through the sample is significant, the present invention provides desirable sensitivity relative to prior art testing devices (e.g., Vitek cards) of which applicant is aware.

FIG. 8 illustrates the absorbance of colors as a function of wavelength to illustrate how the present instrument 20 utilizes selective absorbance to distinguish color changes within a sample.

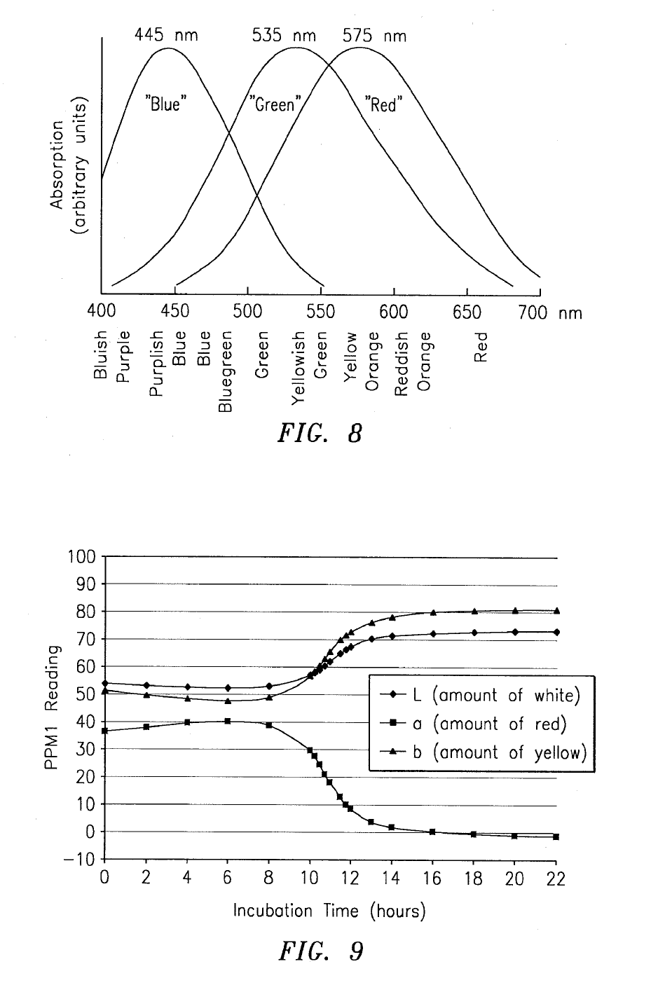

As indicated above, an admixture of the test sample and test mixture will have an initial color and/or degree of transparency. If, for example, a sample admixture is initially transparent, all of the white light produced by the light source 32 will pass through the sample admixture with negligible absorption, and the light detector 34 will collect the white light and produce signals representative of the white light. FIG. 9 illustrates a graph depicting test results of an admixture that initially has a red color. The admixture shown in FIG. 9 includes a test mixture selective to pathogenic staphylococci; e.g., EPI-Mchrom™ selective medium produced by Pilots Point Holdings, LLC of Sarasota, Fla., USA. As can be seen in the graph, light absorbance data for three colors (white, red, and yellow) is depicted along three lines for the entire test period. Initially, the line for each color is substantially linear, extending in a horizontal direction, up until about the eight (8) hour point. During the first eight hours, the data indicates that the test admixture is in the lag phase, and substantial growth of the target organism has not yet occurred within the admixture. In the portion of the graph representative of hours eight through about fourteen, the slope of each of the data lines changes dramatically indicating the target organism log growth phase. In the case of the lines representing the presence of colors yellow and white, the slope of the respective lines increases positively indicating an increase of the respective color. Regarding the color yellow, the increase is attributable to one or more metabolizable substrates within the test mixture being metabolized by the target organism and a color moiety being released within the admixture. The degree to which substrate within the admixture is metabolized and the color moiety released is indicative of the increased presence of target organisms present within the admixture. Regarding the color white (which may also be referred to as “luminescence”), the increase is attributable to the increase in turbidity within the admixture, which increase is attributable to the increase in organism CFUs within the admixture. The decrease in the color red within the admixture reflects the degree to which the initial admixture red color is replaced by the yellow and white coloration. The slope of each color line becomes substantially linear at about the sixteen (16) hour point and remains substantially horizontal until the twenty-two hour point, indicating a stationary growth phase of the target organism. As can be seen in the results shown in FIG. 9, the test produces definitive results in as early as sixteen (16) hours. A test sample with a greater initial amount of target organisms can be expected to produce definitive results in a shorter period of time.

Importantly, aspects of present invention are not limited to detecting the presence or absence of the target organism within a sample admixture, but also can produce semi-quantitative results. For example, the control system may be adapted to interpret the detected color or turbidity/transparency values (or changes) for a given test to produce semi-quantitative information. For example, the control system 36 may be programmed to include color and/or transparency values for a given test, which values are correlated to target organism per volume values. The target organism per volume values may be based on empirical data, and representative values of the empirical data (e.g., values based on a statistically acceptable number of tests) are programmed into the control system 36. In this manner, the control system can provide semi-quantitative information regarding the number (e.g., an average) of target organisms present within a sample admixture. The semi-quantitative representative values can be stored within the control system 36 in a variety of different ways (e.g., look up tables, algorithmic solutions, etc.), and the control system is not limited to any particular type of access to such data.

FIG. 10 illustrates test results wherein the EPI-Mchrom™ selective medium was used to test several different target organism isolates (i.e., MRSA clinical isolates numbers 50153, 43681, 42869, 51667, and 43300) using the present apparatus. As can be seen from the results, the present apparatus correctly identified the presence of the isolates, with each line going from lag phase to log phase growth. These results also indicate the following time to positive result and the quantity of organism CFUs detected for each admixture:

| MRSA Isolate | Time to Positive Result | Total CFU/mL |
|---|---|---|
| Clinical Isolate 50153 | 6.25 hrs | 24,000 |
| Clinical Isolate 43681 | 6.0 hrs | 14,800 |
| Clinical Isolate 42869 | 8.5 hrs | 10,400 |
| Clinical Isolate 51667 | 9.0 hrs | 4,800 |
| ATCC 43300 | 9.75 hrs | 4,000 |

Hence, definitive positive detection was achieved in as little as 6.0 hours.

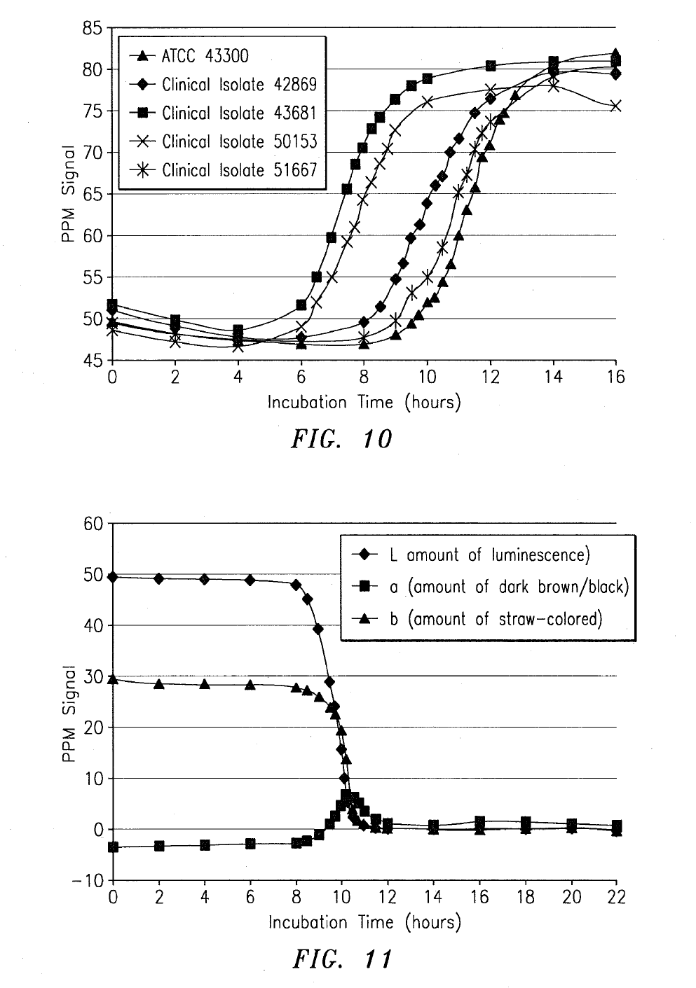

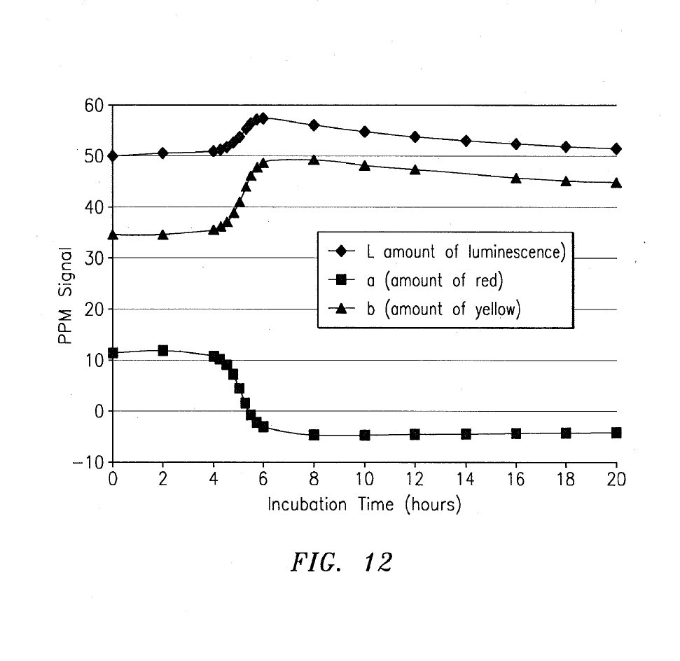

FIG. 11 illustrates a graph depicting test results of an admixture that initially has a straw color with a degree of luminescence. The admixture shown in FIG. 11 includes a test mixture selective to vancomycin-resistant enterococcus (either E. faecalis or E. feacium;—collectively “VRE”); e.g., EPI-V selective medium produced by Pilots Point Holdings, LLC of Sarasota, Fla., USA. As can be seen in the graph, light absorbance (or emittance) data for three colors (white/luminescence, straw, and brown/black) is depicted along three lines for the entire twenty-two hour test period. Initially, the line for each color is substantially linear, extending in a horizontal direction, up until about the eight (8) hour point. During the first eight hours, the data indicates that the test admixture is in the lag phase, and substantial growth of the target organism has not yet occurred within the admixture. In the portion of the graph representative of hours eight through about twelve, the slope of each of the data lines changes dramatically indicating the target organism log growth phase. In the case of the lines representing the presence of colors white/luminesence, the slope of the respective lines decrease negatively indicating a decrease of the respective color. During the same time, the slope of the line representing brown/black increases. Hence, the test admixture changes color from straw/luminescence to brown/black indicate a positive result. As indicated above, the degree to which the indicator color (or degree of transparency) is present within the admixture can be quantified, and therefore the associated quantity of the target organism within the admixture can also be determined. As can be seen in the results shown in FIG. 11, the test produces definitive results in as early as ten (10) hours.

FIG. 12 illustrates a graph depicting test results of an admixture that initially has a red color. The admixture shown in FIG. 12 includes a test mixture selective to carbapenem-resistant Enterobacteriaceae (“CRE”); e.g., EPI-CRE™ selective medium produced by Pilots Point Holdings, LLC of Sarasota, Fla., USA. As can be seen in the graph, light absorbance (or emittance) data for three colors (red, white/luminescence, and yellow) is depicted along three lines for the entire twenty (20) hour test period. Initially, the line for each color is substantially linear, extending in a horizontal direction, up until about the four (4) hour point. During the first four hours, the data indicates that the test admixture is in the lag phase, and substantial growth of the target organism has not yet occurred within the admixture. In the portion of the graph representative of hours four through about six, the slope of each of the data lines changes dramatically indicating the target organism log growth phase. In the case of the lines representing the presence of colors yellow and white/luminesence, the slope of the respective lines increases positively indicating an increase of the respective color. During the same time, the slope of the line representing red decreases. Hence, the test admixture changes color from red to yellow with luminescence indicating a positive result. As indicated above, the degree to which the indicator color is present within the admixture can be quantified, and therefore the associated quantity of the target organism within the admixture can also be quantified (e.g., see the vertical column of the graph). As can be seen in the results shown in FIG. 12, the test produces definitive results in as early as six (6) hours.

Once the analysis information is determined, the control system 36 of the present instrument is adapted to selectively display the results (e.g., on a monitor, via print medium, etc.) and/or send the results to a connected network, or transmit the results to a remote location. In some embodiments, the analysis results may be displayed and/or communicated in real time to give an operator the ability to provide real time analysis of any significant epidemiology changes, and consequent ability to use that information to direct appropriate efforts at critical control points. As indicated above, the results may be an indication of the presence or absence of the target organism, and may also indicate semi-quantitative information regarding the amount of target organism present within the test admixture.

From the above, it can be seen that the present instrument may be used to analyze multiple test samples admixed with the same test mixture, or may be used to analyze multiple samples, some of which may be admixed with different test mixtures. The position on the carousel 28 of a container 24 holding a given admixture can be input to the instrument and the test data specific for the type of analysis can determined; e.g., a carousel 28 having twenty test container 24 positions could be used to perform twenty different types of tests at one time. Each test utilizes the same white light source 32 to produce the desired data.

As indicated above, embodiments of the present instrument include a carousel 28 that moves relative to a stationary light source 32/light detector 34 pair, a light source 32/light detector 34 pair that moves relative to a stationary carousel 28, and a stationary carousel 28 used with stationary light source 32/light detector 34 pairs. The flexibility of the present instrument, as can be seen by these embodiments, permits a wide variety of test containers 24 to be used; e.g., it may be preferable to use a large rotatable carousel 28 that holds a large number of small volume test containers 24 (e.g., test tubes), or it may be preferable to use a stationary carousel 28 for large volume test containers 24 (e.g., 100 mL test containers 24).

A method for detecting the presence or absence of a target organism in a test sample according to an aspect of the present invention may be described as including the steps of: a) providing an instrument having a carousel configured to hold a plurality of test sample containers, with each container held by the carousel at a container position, and each of which containers is operable to hold an admixture of a test mixture inoculated with the test sample, a light source and a light detector paired together, and a control system; b) emitting white light using the light source, and detecting light emitted by the light source using the light detector, which light has traveled along a light axis extending through the test sample disposed within the test container, which light detector is operable to produce signals representative of the light detected by the light detector; and c) producing information indicative of the presence or absence of the target organism in the test sample using a control system to process the signals produced by the light detector.

Although this invention has been shown and described with respect to the detailed embodiments thereof, it will be understood by those skilled in the art that various changes in form and detail may be made without departing from the spirit and scope of the invention.

What is claimed is:

1-25. (canceled)

26. An instrument for detecting the presence or absence of a target organism in a test sample, the instrument comprising:
a carousel configured to hold a plurality of test sample containers, with each container held by the carousel at a container position, and each of which containers having an interior volume configured to hold an admixture of a test mixture inoculated with the test sample;
a drive mechanism;
a light source that emits white light;
a light detector configured to detect the light emitted by the light source, and which light detector is configured to produce signals representative of the light detected by the light detector;
which light detector and light source are aligned with one another along a light axis, and at least a portion of the light produced by the light source travels from the light source towards the light detector along the light axis,
a control system that includes a processor in communication with the drive mechanism, the light source, the light detector, and a machine-readable storage device storing instructions, which instructions when executed cause the control system to selectively operate the drive mechanism to align one of the carousel container positions with the light axis, and cause the light source to emit light along the light axis, at least a portion of which light passes through the aligned carousel container position, and toward the light detector, and which instructions cause the control system to selectively analyze the signals received from the light detector and to produce information indicative of the presence or absence of the target organism in the test sample using signals produced by the light detector.

27. The instrument of claim 26, wherein the light source further emits ultraviolet light.

28. The instrument of claim 26, wherein the light source further emits infrared light.

29. The instrument of claim 26, wherein the instructions when executed cause the control system to determine a change of color in a test sample admixture disposed in at least one of the test sample containers using the signals produced by the light detector.

30. The instrument of claim 26, wherein the instructions when executed cause the control system to determine a change in a level of turbidity in a test sample admixture disposed in at least one of the test sample containers from the signals produced by the light detector.

31. The instrument of claim 26, wherein the carousel and the drive mechanism are configured to produce rotational movement of at least one of the carousel and the light source and light detector relative to the other of the carousel and the light source and light detector.

32. The instrument of claim 26, wherein the carousel and the drive mechanism are configured to produce linear movement of at least one of the carousel and the light source and light detector relative to the other of the carousel and the light source and light detector.

33. The instrument of claim 26, wherein the carousel includes “n” number of container positions, where “n” is an integer, and the instrument includes “n” number of light sources and “n” number of light detectors, with each light source forming a light source/light detector pair with one of the light detectors, with each light source/light detector pairs aligned along a respective light axis extending through one of the container positions, and the carousel, the light sources, and the light detectors are stationary relative to one another.

34. The instrument of claim 26, wherein the instructions when executed cause the light source to periodically emit light along the light axis when a particular one of the carousel container positions is aligned with the light axis during a test period.

35. The instrument of claim 34, wherein the instructions when executed cause the control system to produce the information indicative of the presence or absence of the target organism for a given one of the test samples for each of a plurality of intervals during the test period.

36. The instrument of claim 35, wherein the instructions when executed cause the control system to produce semi-quantitative information representative of a per volume number of CFUs of the target organism within the test sample.

37. The instrument of claim 35, wherein the instructions when executed cause the control system to create a growth curve for the test period using the information indicative of the presence or absence of the target organism for the given one of the test samples at each periodic interval of time.

38. The instrument of claim 26, wherein the instructions when executed cause the control system to produce information representative of a per volume number of CUs of the target organism within the test sample.

39. A method for detecting the presence or absence of a target organism in a test sample, the method comprising the steps of:
providing an instrument having a control system that includes a processor in communication with a machine-readable storage device storing instructions, at least one light source and light detector pair, and a carousel configured to hold a plurality of test sample containers, with each container held by the carousel at a container position, and each of which containers is configured to hold an admixture of a test mixture inoculated with the test sample;
emitting white light using the light source along a light axis that extends through test sample disposed within an aligned one of the test containers, and detecting at least a portion of the light emitted by the light source using the light detector, and producing signals representative of the light detected by the light detector; and
producing information indicative of the presence or absence of the target organism in the test sample using the instructions executed by the control system to selectively analyze the signals received from the light detector.

40. The method of claim 39, wherein the step of producing information includes at least one of determining a change of color in the admixture or determining a change in a level of turbidity within the admixture.

41. The method of claim 40, further including the step of moving one of the carousel and the light source and light detector pair, relative to the other, so that each container position is alignable with the light source and light detector pair.

42. The method of claim 41, wherein the step of emitting white light using the light source, and detecting light emitted by the light source is performed at periodic intervals of time over a test period.

43. The method of claim 42, wherein the step of producing information is performed for each periodic interval of time.

44. The method of claim 43, wherein the step of producing information includes producing a value representative of the per volume number of CFUs of the target organism within the test admixture.

45. The method of claim 44, further including the step of producing a growth curve for the test period using the information produced at each periodic interval of time.
